(19) United States
(12) Patent Application Publication
(10) Pub. No.: US 2011/0136909 A1
(43) Pub. Date: Jun. 9, 2011

(54) METHOD FOR SUPPRESSING EXCESSIVE APPETITE

(76) Inventors: Toshifumi Imada, Kawasaki-shi (JP); Shuzhen Hao, Palisades Park, NJ (US); Tatsuro Tanaka, Kawasaki-shi (JP); Miki Tomoe, Kawasaki-shi (JP); Eiichiro Kimura, Kawasaki-shi (JP); Hisayuki Uneyama, Kawasaki-shi (JP); Kunio Torii, Kawasaki-shi (JP)

(21) Appl. No.: 12/972,670

(22) Filed: Dec. 20, 2010

Related U.S. Application Data
(63) Continuation-in-part of application No. PCT/JP2009/061239, filed on Jun. 19, 2009.

(30) Foreign Application Priority Data
Nov. 17, 2008 (JP) 2008-293572
Jun. 19, 2009 (JP) 2008-161012

(51) Int. Cl. A61K 31/198; A61P 3/04; A61P 3/00

(52) U.S. Cl. 514/561

(57) ABSTRACT

Agents for the inhibition, suppression, or prevention of overeating include glutamic acid and/or at least one of its salts, as is or in a carrier or base, and have superior safety at a low price and further have a favorable flavor and are suited for long-term use. Such agents will amplify or sustain a sense of fullness and, further, are effective against appetite stimulation that arises regardless of a sense of fullness with respect to foods that are, for example, sweet or delicious, especially for a person who has a drive for thinness.

This application is a Continuation-in-Part of, and claims priority under 35 U.S.C. §120 to, International Application No. PCT/JP2009/061239, filed 19 Jun. 2009, and claims priority therethrough under 35 U.S.C. §§119, 365, to Japanese Application Nos. 161012/2008, filed 19 Jun. 2008, and 293572/2008, filed 17 Nov. 2008, the entireties of which are incorporated by reference herein.

### BACKGROUND

1. Field of Endeavor

This invention relates to agents for the inhibition, suppression, or prevention of overeating that have the effect of amplifying a sense of fullness or satiety, or sustaining the effect or of inhibiting appetite stimulation that arises regardless of whether one feels full with respect to sweets or delicacies or one has enough calorie intake for maintaining daily energy expenditure. Agents for the inhibition, suppression, or prevention of overeating related to this invention can be used for the treatment of binge eating as well as preventing or remedying obesity.

2. Brief Description of the Related Art

Eating is an essential behavior to acquire the requisite energy to sustain life and for biological activities. Animals generally begin eating when they have the feeling of hunger and when their satiety center in the brain is stimulated through sufficient consumption of food, they stop their eating. However, the sense of hunger arises due to a variety of factors and, in addition to consumption hormones, substances that act on the appetite center and the satiety center are involved.

In recent years, overeating due to stress and disease has become a problem. Furthermore, obesity is becoming a major social problem due to dietary habits becoming more like those of the U.S. and Europe and due to insufficient exercise. Obesity has become the source of the three major lifestyle illnesses of hypertension, hyperlipidemia, and diabetes and thus preventing or remedying obesity is very important to maintaining a healthy life, and amplifying or sustaining a sense of fullness and controlling consumption volume will be useful in preventing or remedying obesity. On the other hand, the consumption of appropriate nutrients is also necessary for maintaining life activities and for living a physically and mentally healthy life. In other words, preventing overeating is important for preventing and remedying obesity. In clinical conditions, preventing and remedying obesity is not easy. Although there are three common approaches to curing obesity, including administration of appetite suppressing drugs, surgical approaches, and behavioral approaches, these are very costly and burdensome for the patients. Furthermore, it is very difficult to prevent weight rebound with overeating and obesity, because it is difficult to provide feelings of fullness or satiety while keeping a restricted calorie intake.

Substances that amplify or sustain a sense of fullness to prevent overeating have been developed and those including dietary fiber that has been ground into microgranules (see Japanese Unexamined Patent Application Publication No. 2005-58093); those including a mixture of two or more water soluble polysaccharides such as xanthan gum, konjac, or locust bean gum (see Japanese Unexamined Patent Application Publication No. 2002-218943); those including potato extracts as protease inhibitors (see Japanese Unexamined Patent Application Publication No. 2005-336208), and others have been disclosed.

Nevertheless, with the above-noted substances that amplify or sustain a sense of fullness, it is not possible to make a meal more enjoyable because the taste and texture of the meal are secondary, although effects are achieved in the reduction of the amount eaten. In addition, controlling amounts eaten by amplifying or sustaining a sense of fullness must be implemented over a considerably long period in order to cure binge eating or prevent or remedy obesity. However, conventional products have high costs and side effects are seen, such as diarrhea, depending on the nature of the substance. Thus it is difficult to say that they are suitable for long-term use. Furthermore, we generally have available to us many sweets and delicacies such as desserts that come after a meal and sweets eaten in between meals and so people end up eating foods such as sweets, even if they have a sense of fullness, a phenomenon dubbed an “extra stomach.” As a result, people frequently end up overeating. In this phenomenon, it has been suggested that a consumption-stimulating effect mediated by activation of an appetite-stimulating neuropeptide due to sweet flavors and good flavors (see Brain Res. 175 (2) 241-248 (2006)) is involved, in addition to sensory-specific satiety. Accordingly, there is a possibility that we cannot prevent the overeating of desserts, sweets, and the like after meals and in between meals simply by amplifying and sustaining a sense of fullness.

### SUMMARY

One of numerous aspects of the present invention includes substances that are agents for the inhibition, suppression, or prevention of overeating that are low priced and superior in safety and that can further impart favorable flavor and are suitable for long-term use and that amplify or sustain a sense of fullness and are useful against appetite stimulation that arises regardless of a sense of fullness with respect to foods that are, for example, sweet and delicious.

A thorough examination was made in order to resolve the above-noted problems and as a result, the inventors discovered that when glutamic acid and/or its salts are consumed, they have a superior action in the prevention of overeating by amplifying or sustaining a sense of fullness and inhibiting appetite stimulation that arises regardless of the sense of fullness with respect to foods that are, for example, sweet or delicious, especially for a person who has a drive for thinness.

Specifically, several other aspects of the present invention concern the following:

(1) Agents for the prevention of overeating that include glutamic acid and/or at least one type of its salts.

(2) Agents for the inhibition, suppression, or prevention of overeating described in the above-noted (1), in which each intake of the glutamic acid and/or at least one of its salts is between 0.01 g-100 g, converting to free glutamic acid, in an adult human.

(3) Agents for the inhibition, suppression, or prevention of overeating that include an umami taste receptor agonist and at least one type of its salts.

(4) Agents for the inhibition, suppression, or prevention of overeating described in any of the above-noted (1)-(3) that are agents that amplify or sustain a sense of fullness or satiety.

(5) Agents for the inhibition, suppression, or prevention of overeating described in any of the above-noted (1)-(3) that are agents to inhibit appetite stimulation that arises regardless of a sense of fullness.

(6) Methods of use of glutamic acid and/or at least one type of its salts for the purpose of manufacturing agents for the prevention of overeating.

(7) Methods of use of glutamic acid and/or at least one type of its salts for the purpose of manufacturing agents to amplify or sustain a sense of fullness or satiety.

(8) Methods of use of glutamic acid and/or at least one type of its salts for the purpose of manufacturing agents to inhibit appetite stimulation that arises regardless of a sense of fullness.

(9) Methods of use of an umami taste receptor agonist and/or at least one type of its salts for the purpose of manufacturing agents for the prevention of overeating.

(10) Methods of use of an umami taste receptor agonist and/or at least one type of its salts for the purpose of manufacturing agents to amplify or sustain a sense of fullness.

(11) Methods of use of an umami taste receptor agonist and/or at least one type of its salts for the purpose of manufacturing agents to inhibit appetite stimulation that arises regardless of a sense of fullness.

(12) A therapeutic method for treating binge eating including administration of glutamic acid and/or at least one of its salts in a sufficient amount to prevent overeating, to subjects in which preventing overeating is required.

(13) The therapeutic method described in the above-noted (12) in which the sufficient amount to prevent overeating per administration is between 0.01 g-100 g, converting to free glutamic acid, in a human adult.

(14) A method to prevent or remedy obesity including administration of glutamic acid and/or at least one of its salts to subjects in which preventing or remedying obesity is required in a sufficient amount to achieve said effects.

(15) The method to prevent or remedy obesity described in the above-noted (14) in which the sufficient amount to achieve the effect of preventing or remedying obesity per administration is between 0.01 g-100 g, converting to free glutamic acid, in a human adult.

(16) A therapeutic method for addressing binge eating including the administration of an umami taste receptor agonist and/or at least one of its salts in an amount sufficient to prevent overeating to subjects in which preventing overeating is required.

(17) A method for preventing or remedying obesity that includes administration of an umami taste receptor agonist and/or at least one of its salts to subjects in which preventing or remedying obesity is required in a sufficient amount to achieve said effects.

(18) Agents for the prevention of overeating described in any one of the above-noted (1)-(3) and commercial packaging including documents that describe that the agent for the prevention of overeating can be used or should be used in order to treat binge eating or prevent or remedy obesity.

(19) A method for suppressing overeating, said method comprising ingesting at least one substance selected from the group consisting of glutamic acid and a salt thereof, in an amount sufficient to suppress overeating, by a person who needs suppression of overeating.

(20) The method according to (19), wherein said sufficient amount for suppressing overeating is between 0.01 g to 100 g as a free glutamic acid, per ingestion for an adult human.

(21) The method according to (19), further comprising:
adding between 0.01 g to 100 g of said substance per ingestion to a food or drink;
wherein said ingesting comprises ingesting said food or drink.

(22) The method according to (21), wherein said adding comprises adding said substance to a concentration of between 0.0001% to 10% by weight as a free glutamic acid in said food or drink.

(23) The method according to (19), wherein said person is a person who has drive for thinness as indicated by their EDI Section A score.

(24) The method according to (19), wherein said person has a non-zero EDI Section A score.

(25) The method according to (19), further comprising identifying said person prior to said ingesting as a person who agrees with at least one statement having a meaning substantially the same as at least one of following statements:
(i) I eat sweets and carbohydrates without feeling nervous;
(ii) I think about dieting;
(iii) I feel extremely guilty after overeating;
(iv) I am terrified of gaining weight;
(v) I exaggerate or magnify the importance of weight;
(vi) I am preoccupied with the desire to be thinner; and
(vii) If I gain a pound I get worried that I will keep gaining.

(26) The method for controlling or improving obesity, the method comprising suppressing overeating according to the method of (19).

(27) The method for suppressing overeating by a patient who overeats, the method comprising prescribing to the patient medical food comprising:
(A) at least one substance selected from the group consisting of glutamic acid and a salt thereof; or
(B) a food or drink to which at least one substance selected from the group consisting of glutamic acid and a salt thereof has been added.

(28) The method according to (27), wherein said medical food is:
(A) 0.01 g to 100 g of at least one substance selected from the group consisting of glutamic acid and a salt thereof, as a free glutamic acid, per ingestion by a adult human; or
(B) a food or drink to which 0.01 g to 100 g of at least one substance selected from the group consisting of glutamic acid and a salt thereof has been added, as a free glutamic acid, per ingestion by an adult human.

(29) The method according to (27), wherein said medical food comprises said substance in a concentration of 0.0001% to 10% by weight as a free glutamic acid.

(30) The method according to (27), wherein said prescribing comprises prescribing said medical food to a person who has drive for thinness as indicated by their EDI Section A score.

(31) The method according to (27), wherein said person has a non-zero EDI Section A score.

(32) The method according to (27), further comprising identifying said patient prior to said prescribing as a person who agrees with at least one statement having a meaning substantially the same as at least one of the following statements:
(i) I eat sweets and carbohydrates without feeling nervous,
(ii) I think about dieting,
(iii) I feel extremely guilty after overeating,
(iv) I am terrified of gaining weight,
(v) I exaggerate or magnify the importance of weight,
(vi) I am preoccupied with the desire to be thinner, and
(vii) If I gain a pound I get worried that I will keep gaining.

(33) A method for controlling or improving obesity, said method comprising suppressing overeating according to the method of (27).

With products and methods embodying principles of the present invention, it is possible to provide agents for the inhibition, suppression, or prevention of overeating that make it possible to effectively prevent overeating by effectively amplifying or sustaining a sense of fullness or controlling appetite stimulation with respect to foods that are sweet or delicious, regardless of a feeling of fullness, which is known as having a so-called “dessert stomach.” Agents for the inhibition, suppression, or prevention of overeating embodying principles of the present invention have superior safety at a low price and, further, can provide a favorable flavor and are appropriate for long-term use for the purpose of treating binge eating and for preventing or remedying obesity.

In the context of the subject matter herein, “fullness” refers to a sensation experienced by a person after eating, the sensation being of a state of having eaten enough or more than enough and feeling full. In the context of the subject matter herein, “satiety” refers to a sensation experienced by a person after eating, the sensation being of a state that eating is suppressed or inhibited for a certain time.

### BRIEF DESCRIPTION OF THE DRAWINGS

FIG. 1 is a diagram showing changes over time in the sense of fullness for a group that consumed the agent to amplify the sense of fullness of Working Example 1 and a group that consumed the food of Comparison Example 1.

FIG. 2 is a diagram showing changes over time in the sense of fullness for a group that consumed the agent to amplify a sense of fullness of Working Example 2 and a group that consumed the food of Comparison Example 2.

FIG. 3 is a diagram showing desert consumption amount after eating (satiety effect) for a group that consumed the agent to prevent overeating of Working Example 3 and a group that consumed the food of Comparison Example 3.

FIG. 4 is a diagram showing participants' likability of a placebo soup or soups with additions of MSG alone or MSG+IMP in a study.

FIG. 5 is a diagram showing participants' perception of fullness over time after the ingestion of placebo soups, and soup with the addition of MSG alone or MSG+IMP.

FIG. 6 is a diagram showing preloaded soup type versus calorie intake for participants in a study (satiety effect).

FIG. 7A is a diagram showing the negative association between the scores of the Eating Disorder Index (EDI) versus difference of calorie intake between soups with the addition of MSG and placebo (satiety effect).

FIG. 7B is a diagram showing the negative association between the scores of the Eating Disorder Index (EDI) versus difference of calorie intake between soups with the addition of MSG+IMP and placebo (satiety effect).

### DETAILED DESCRIPTION OF EXEMPLARY EMBODIMENTS

Herein, agents for the inhibition, suppression, or prevention of overeating include glutamic acid and at least one type of its salts and these are provided as is or in a pharmaceutical or food or beverage carrier or base. For the glutamic acid, either an L-type, D-type, or DL-type can be used, but the L-type can be used preferably.

For the salt of glutamic acid, there are no restrictions in particular, so long as it can be provided as a pharmaceutical product or food or beverage product and inorganic salts such as alkaline metal salts including sodium salts and potassium salts, alkaline earth metal salts including magnesium salts and calcium salts, and ammonium salts and organic salts such as basic amino acid salts including arginine salts, lysine salts, and ornithine salts; amine salts including monoethanolamine salts and diethanolamine salts; and pyrimidine salts, can be noted as examples and from among these, sodium salts are to be used preferably given their superior solubility in water and flavor. In addition, for the glutamic acid and its salts, it is possible to use natural substances that are derived from animal and vegetable raw materials as well as that which is obtained through chemical synthesis, fermentation, genetic recombination, or the like and further, it is possible to purchase commercially available product from, for example, Sigma-Aldrich Corp. and to use that.

With agents for the inhibition, suppression, or prevention of overeating related to this invention, the glutamic acid and/or at least one type of its salts, may be administered as they are or may be administered together with various additives and the like. Furthermore, formulations may be made by using various carriers or bases and additives and various formulations such as drug formulations and food and beverage formulations are acceptable. Agents for the inhibition, suppression, or prevention of overeating related to this invention are suited, in particular, for formulations that are administered orally.

With agents for the inhibition, suppression, or prevention of overeating related to this invention, in order to obtain overeating inhibition, suppression, or prevention effects, it is desirable that the glutamic acid and/or the at least one of its salts be at between 0.01 g-100 g in a free glutamic acid conversion per single intake in a human adult and it is further desirable that it be at between 0.1 g-10 g. Thus, it is desirable that it be administered such that this amount can be consumed or provided included in a carrier or base.

It is possible to provide forms such as aqueous solutions, oily solutions, emulsions, gels, powders, granules, pills, tablets, capsules, and the like by using aqueous, oily, emulsion-form, gel-form, powder-form, and other carriers and bases. In addition, the agents for the inhibition, suppression, or prevention of overeating related to this invention may be, as described below, combined in foods and beverages and, further, formulations may be made in which the glutamic acid and/or at least one of its salts themselves or products containing those are combined in such foods and beverages.

In addition, it is possible to include additives including filling agents such as cellulose and its derivatives including crystal cellulose, hydroxypropyl cellulose; starch and its derivatives including wheat starch, corn starch, sodium carboxymethyl starch, and dextrin; natural high-polymer compounds such as gum arabic and sodium alginate; sugars and their derivatives including glucose, maltose, sorbitol, maltitol, and mannitol; and inorganic salts including sodium chloride, calcium carbonate, and magnesium silicate; binding agents such as guar gum, synthetic aluminum silicate, stearic acid, highly-polymer polyvinylpyrrolidone, and lactic acid; lubricants such as talc, magnesium stearate, and polyethylene glycol 6000; disintegrating agents such as adipic acid, calcium stearate, and white sugar; surface acting agents such as sucrose fatty acid esters, soybean lecithin, polyoxyethylene hydrogenated castor oil, and polyoxyethylene monostearate esters; thickening agents such as sodium carboxymethyl cellulose, carboxyvinyl polymers, xanthan gum, and gelatin; coating agents such as ethyl acrylate methyl methacrylate copolymer dispersion, caramel, carnauba wax, shellac, white sugar, and pullulan; pH adjusting agents such as citric acid, sodium citrate, acetic acid, sodium acetate, and sodium hydroxide; antioxidant agents such as ascorbic acid, tocopherol acetate, natural vitamin E, and propyl gallate; flavorings such as aspartame, licorice extract, and saccharin; preservatives, such as sodium benzoate, sodium edetate, sorbic acid, sodium sorbate, methyl parahydroxybenzoate, and butyl parahydroxybenzoate; and coloring agents such as red iron oxide, yellow iron oxide, black iron oxide, carmine, Food Blue No. 1, Food Yellow No. 4, Food Yellow No. 4 Aluminum Lake, Food Yellow No. 2, and sodium copper chlorophyllin.

It is possible to obtain food and beverage formulations that make it possible to inhibit or prevent overeating by including, in a food-and-beverage-use carrier or base, glutamic acid and/or at least one of its salts. The food and beverage products that have the effect of preventing overeating related to this invention may use any of a solid form, a semi-solid form, or a liquid form. In addition, the aforementioned food and beverage formulations may be provided as foods for specified health use, foods with health claims such as foods with nutrient function claims, and nutritional supplements.

Agents for the inhibition, suppression, or prevention of overeating related to this invention may also be that made by including glutamic acid and/or at least one of its salts in an existing food or beverage product having a small amount of these.

For the carriers or bases or existing food and beverage products in which glutamic acid and/or at least one of its salts are included, those with total contents of glutamic acid and their salts of under 0.02 weight % are to be made to contain glutamic acid and/or at least one of its salts typically at 0.02 weight % or greater, converting it to free glutamic acid. The upper limit for the content is preferably 5 weight % or below, and more preferably is 1 weight % or below.

In addition, it is possible to include an umami taste receptor agonist that includes glutamic acid and/or salts of glutamic acid, and/or at least one of the agonist's salts. For umami taste receptor agonists other than glutamic acid and its salts, nucleic acid flavoring compounds and the like such as 5′-inosinic acid, 5′-guanylic acid, and 5′-adenylic acid can be noted and, for their salts, inorganic salts such as alkaline metal salts including sodium salts and potassium salts; alkaline earth metal salts including magnesium salts and calcium salts; and ammonium salts and organic salts of basic amino acid salts including arginine salts, lysine salts, and ornithine salts; amine salts such as monoethanolamine salts and diethanolamine salts, and pyrimidine salts, can be noted as examples. For these umami taste receptor agonists and their salts other than glutamic acid and its salts as well, it is possible to use natural substances that are derived from animal and vegetable raw materials as well as substances obtained through chemical synthesis, fermentation, genetic recombination, and the like and to use commercially available products. Regarding the content of the flavoring receptor agonist and/or at least one of its salts other than the glutamic acid and its salts, it depends on the type of umami taste receptor agonist and salts used, but between about 0.01 weight %-5 weight % is appropriate.

It is also possible to make a food or beverage formulation by including glutamic acid and/or at least one of its salts, or an umami taste receptor agonist and/or at least one of its salts in, ideally, a solid food such as white rice, bread, chocolate, gum, candy, and cake; a semi-solid food such as rice porridge, oatmeal, tofu, jelly, and pudding; a liquid such as a formula, milk, and cola; and an alcoholic beverage such as beer, beer-like beverages such as low-malt beer, wine, and distilled spirits such as shochu, whiskey, and brandy.

In addition, in cases in which the agent is provided as a food or beverage, it can be a food for specified health use, foods with health claims such as foods with nutrient function claims, nutritional supplements and medical foods. The glutamic acid and/or at least one of its salts, or the umami taste receptor agonist and/or at least one of its salts, can be presented in a form in which the consumption unit volume per time is packed or filled in a bag, box, or the like in an amount to be eaten in one meal or in a form in which a beverage, in which glutamic acid and/or at least one of its salts or the like are suspended or dissolved, is filled in a bottle or the like in a form to be consumed in one meal.

Agents embodying principles of the present invention can be provided as agents to amplify or sustain a sense of fullness or satiety. Agents to amplify or sustain a sense of fullness or satiety can be provided as various formulations as noted above, such as pharmaceutical formulations and food and beverage formulations and it will be possible to effectively amplify or sustain a sense of fullness.

Agents embodying principles of the present invention are also related to inhibiting agents for appetite stimulation that arises when full. In particular, they are substances that will inhibit appetite stimulation that arises regardless of the sense of fullness with respect to foods that are sweet or appetizing and it will be possible to effectively inhibit appetite stimulation in which a person ends up eating sweets and the like regardless of a sense of fullness, which is known as having a so-called “dessert stomach.”

Furthermore, agents embodying principles of the present invention can be added to feeds for animal use.

In addition, methods embodying principles of the present invention for the treatment of binge eating include the administration of glutamic acid and/or at least one of its salts, or an umami taste receptor agonist and/or at least one of its salts, at an amount sufficient to inhibit or prevent overeating to patients or animals in which it is necessary to prevent overeating. As stated previously, for glutamic acid and/or its salts, a sufficient amount to prevent overeating for is between 0.01 g-100 g for a human adult, per administration, converted to free glutamic acid.

In addition, methods to prevent or remedy obesity embodying principles of the present invention include the administration of glutamic acid and/or at least one of its salts, or an umami taste receptor agonist and/or at least one of its salts, at an amount sufficient to obtain the effects of preventing or remedying obesity to patients and animals for which preventing or remedying obesity is necessary. A sufficient volume in order to obtain the effects of preventing or remedying obesity is the same as that noted above for glutamic acid and/or at least one of its salts.

Furthermore, it is possible to provide commercial packaging including documents describing any of the agents noted herein and whether the concerned agent for the prevention of overeating can be used or should be used to treat binge eating or to prevent or remedy obesity.

Because agents for the inhibition, suppression, or prevention of overeating in accordance with the present invention are low priced and have superior safety and, further, can be used to impart good flavor, they are suitable for use over a long term. There are cases in which it would be desirable to use an agent for the inhibition, suppression, or prevention of overeating related to this invention over a considerably long period for the treatment of binge eating and, in particular, for preventing and remedying obesity. The administration period for such agents is to be determined based on factors such as the symptoms and dietary habits of patients and others who are given the substance, but typically, it would be 2 weeks to 60 years, and ideally 1 month to 2 years.

### WORKING EXAMPLES

Further detailed explanation will be provided through working examples.

### Working Example 1

### Liquid-Form Agent to Amplify a Sense of Fullness

Preparation of a liquid carrier was made by eliminating natural flavorings including sodium glutamate, nucleic acid flavors, and yeast extract from a commercially available corn potage. To the aforementioned liquid carrier, sodium L-glutamate was added so as to make it 0.5 weight % and this was taken as a liquid agent to amplify a sense of fullness.

### Working Example 2

### Semi-Solid Agent to Amplify a Sense of Fullness

450 g of a retort-pouch rice porridge made by Ajinomoto Co., Inc., (glutamic acid content: under 0.02 weight %) was heated in water at 53° C. for 30 minutes and sodium L-glutamate was added so that it would be 0.5 weight % and this was taken as a semi-solid agent to amplify a sense of fullness.

Study Case 1

A subjective abdominal sensation evaluation, including the sense of fullness, was made for the above-noted Working Example 1 and Working Example 2. At that time, Comparison Example 1 and 2 foods were prepared by adding table salt such that the sodium concentrations would be equal, instead of sodium L-glutamate, to each of the agents to amplify a sense of fullness of Working Example 1 and Working Example 2.

In the above-noted evaluations, men aged 40 years and older were taken as the subjects and the study was conducted as a double-blind crossover study with groups having 11 subjects each. The subjects were given the study foods after 12 hours of fasting and an evaluation was made of abdominal sensations over a period of 4 hours after consumption. 400 g of each of the above-noted working example agents to amplify a sense of fullness and comparison example foods was measured out and these were taken as the study foods.

In the subjective abdominal sensation evaluation, subjects were asked to evaluate sensations every 15 minutes after consumption of the study foods using a Visual Analog Scale (VAS) and an analysis was made of the data through an analysis of covariance using a linear mixed model. For the subjective abdominal sensations, the categories of sense of emptiness, sense of fullness, stomach bloating, and stomach heaviness were given. The length of the VAS was set at 10 cm and it was set for each of the above-noted evaluation categories as indicated below. Measurement was made of the length from the left edge of the scale to the area checked by the subject and evaluation results were indicated by that length (cm).

VAS

Sense of emptiness:
None at all (0 cm)______Very strong (10 cm)

Sense of fullness:
None at all (0 cm)______Very strong (10 cm)

Stomach bloating:
None at all (0 cm)______Very strong (10 cm)

Stomach heaviness:
None at all (0 cm)______Very strong (10 cm)

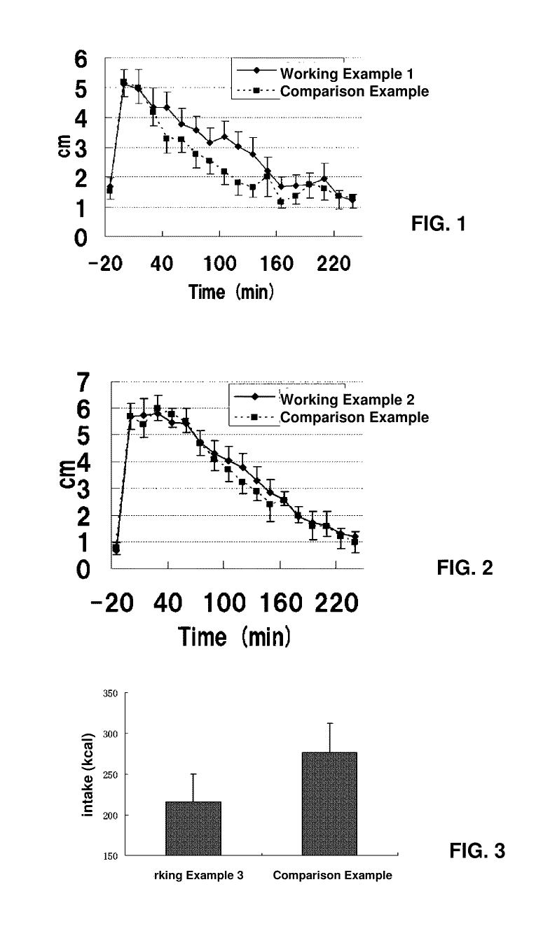

Results of the subjective abdominal sensation evaluation are shown in FIG. 1 and FIG. 2 as changes over time in responses for sense of fullness.

As is evident from FIG. 1, in the group that consumed the agent to amplify a sense of fullness of Working Example 1 of the present invention, the sense of fullness was maintained significantly longer in comparison to the group that consumed the food of Comparison Example 1 (p<0.001). In addition, in the group that consumed the agent to amplify a sense of fullness of Working Example 1, a decline in sense of emptiness (p<0.1) and an increase in stomach bloating (p<0.001) were observed in comparison to the group that consumed the food of Comparison Example 1.

Next, as is evident from FIG. 2, in the group that consumed the agent to amplify a sense of fullness of Working Example 2 of this invention, a continuation of the sense of fullness was observed in comparison to the group that consumed the food of Comparison Example 2. Additionally, in the group that consumed the agent to amplify a sense of fullness of Working Example 2, the evaluation was high for stomach heaviness after consumption of the study food in comparison to the group that consumed the food of Comparison Example 2 (p<0.001).

The results above indicate that in the groups that consumed the agents to amplify a sense of fullness of the working examples of this invention, maintenance of a sense of fullness and a delay in the appearance of a sense of emptiness were observed.

### Working Example 3

### Liquid-Form Agent for the Inhibition, Suppression, or Prevention of Overeating

Preparation of a liquid carrier was made by eliminating natural flavorings including sodium L-glutamate, nucleic acid flavors, and yeast extracts from a commercially available chicken consommé soup. To the aforementioned liquid carrier, sodium L-glutamate was added so as to make it 0.5 weight % and this was taken as a liquid agent for the prevention of overeating.

Study Case 2

An examination was made of the consumption amount of dessert on a full stomach after eating, for the liquid-form agent for the inhibition, suppression, or prevention of overeating of the above-noted Working Example 3. At that time, Comparison Example 3 food was prepared by adding table salt such that the sodium concentration would be equal, instead of adding sodium L-glutamate to the liquid-form agent for the inhibition, suppression, or prevention of overeating of Working Example 3.

The above-noted evaluation of the consumption amount of dessert was conducted as a 12-person double-blind crossover study. In the study, 2 patterns were implemented, with one from 11:30 and another from 12:30 and each subject was required to participate three times. On the study participation days, subjects were given a breakfast of the same content (ordinary breakfast) at the same time all three times.

In the 1^{st} trial of the study, subjects filled in a questionnaire concerning physical condition and the like and were then allowed to freely eat a lunch prepared in advance (consisting of white rice, hamburgers, cucumbers, tomatoes, mashed pumpkin, mashed potatoes, and plain yogurt) and their consumption amounts were measured. In the 2^{nd} and 3^{rd} trials, subjects ate a lunch of the same amount as the 1^{st} trial (except for the plain yogurt) and then were given the study food for consumption in a 2-minute period. Thereafter, they were allowed to freely eat chocolate-flavored, strawberry-flavored, and green-tea-flavored cake and a cream-layer cake and the consumption amount of that was measured. Note that in both the 2^{nd} and 3^{rd} trials, 150 mg of each the liquid-form agent of Working Example 3 and the food of Comparison Example 3 were provided as the respective study foods.

FIG. 3 shows the dessert consumption amount after consumption of the liquid-form agent of Working Example 3 and the food of Comparison Example 3.

As is evident from FIG. 3, the consumption amount of dessert for the group that consumed the liquid-form agent of Working Example 3 was significantly lower (p=0.017) than that of the group that consumed the food of Comparison Example 3 according to a paired t-test.

The results above suggest that it is possible to inhibit or prevent overeating that arises due to appetite stimulation by sweets or delicacies on a full stomach after eating lunch by consuming the liquid-form agent of Working Example 3. The data indicates that MSG provides a superior effect of satiety.

Case Study 3:

Subjects: A total of 20 female subjects aged 30-45 years with normal weight (BMI 18.5-25.0) and general good health who met the study criteria participated in the study. The major exclusion criteria are subjects who have dysgeusia and/or abnormal eating habits. Subjects visited the study site one time per week for three consecutive weeks (cross-over study) for test product (three test products, one test product per visit). Subjects were randomized for the order of test product administration.

Test products: 1) placebo, 200 ml soup containing 0.16% NaCl (equivalent in Na), 2) MSG, 200 ml soup containing 0.5% MSG, and 3) MSG+nucleotide, 200 ml soup containing 0.5% MSG and 0.05% nucleic acid (IMP).

Study procedures: On the visiting day, subjects arrived at the study site at 11:30 am before lunch. A satiety motivational questionnaire was taken immediately before lunch. The subjects had a standard lunch (400 calorie) and 12 ounces water at 12:00 pm. The subjects were instructed to finish the lunch within 30 min. The subjects were asked to consume the entire lunch and water provided. The subjects were instructed to fill out the satiety motivational questionnaire immediately after lunch. Two (2) hours after the lunch, subjects were instructed to fill out the satiety motivational questionnaire and given 200 ml consommé soup (in a bowl with a spoon and napkin, at a temperature of 65° C.) containing either the test product (0.5% MSG or 0.5% MSG and Nucleic Acid) or placebo product (NaCl 0.16%). Subjects were instructed to finish eating the soup in 10 minutes, and filled out the soup rating questionnaire and the satiety motivational questionnaire immediately after consuming the soup. Fifteen (15) minutes after the test product administration, subjects were given a snack and water ad lib, and the amounts of intake were recorded. The snacks presented to the subjects include four types: 1) high fat savory group including cheddar cheese, PRINGLES original, dry roasted peanut-salted, Meza hummus, 2) low fat savory group including reduced fat biscuits, sugar snap peas, KRONOS pita bread, hard pretzels, 3) high fat sweet group including M&M ‘fun pak’, KLONDIKE ice cream bar, jelly filled raised doughnut, and 4) low fat sweet group include apple dippers, RICE KRISPIES TREATS, mixed berry yogurt (YOPLAIT orig., 99% fat free), baby carrots and banana. Subjects were instructed to eat as much as or as little as they want. Subjects were instructed to fill out a satiety questionnaire immediately after snack testing and at 3:15 pm and 4:00 pm. The subjects were prohibited from exercising and asked not to consume any food or water until soup serving.

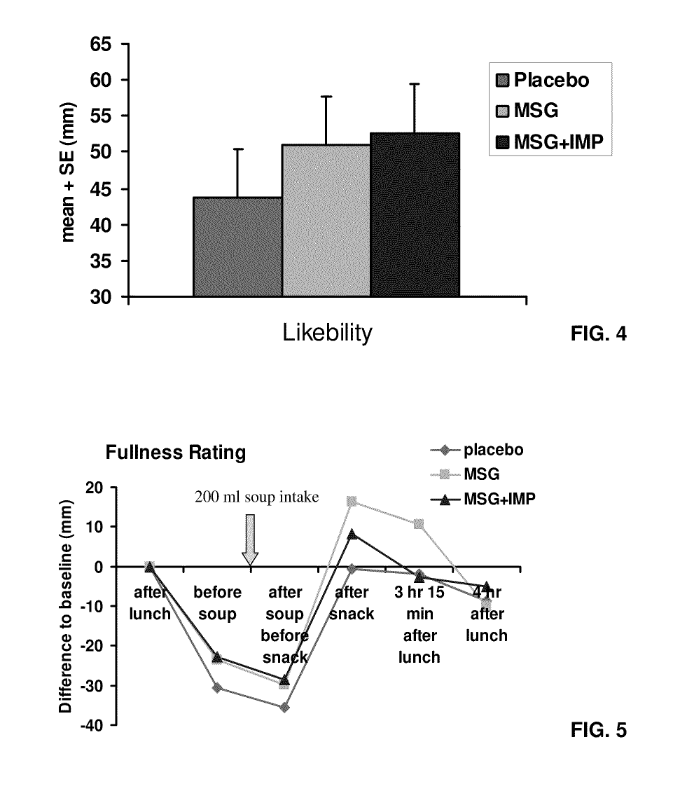

FIG. 4 demonstrates that participants liked more of the soups with the addition of umami substances (MSG, MSG+IMP) than a placebo soup. The data of FIG. 4 was obtained in case study 3. The study participants estimated “likability” of the three types of soups after eating them according to VAS:(Dislike extremely(0 cm) Like extremely (10 cm). The vertical scale of FIG. 4 shows the VAS value, in which the units have been converted to “mm”. The data of FIG. 4 indicates that MSG imparts favorable flavor and is suitable for long-term use.

FIG. 5 demonstrates that participants who ingested the soups with the additions of umami substances (MSG, MSG+IMP) felt more fullness in comparison to the placebo soup ingestion. The data of FIG. 5 was obtained in case study 3. The people estimated the three types of soups according to VAS for “fullness” (Not full at all (0 cm) to extremely full (10 cm). In FIG. 5, the results of the estimate is shown as the difference of VAS value, that is, subtracted VAS values at after lunch (baseline) from VAS values at each estimate time. The units of the vertical scale of FIG. 5 has been converted to “mm”.

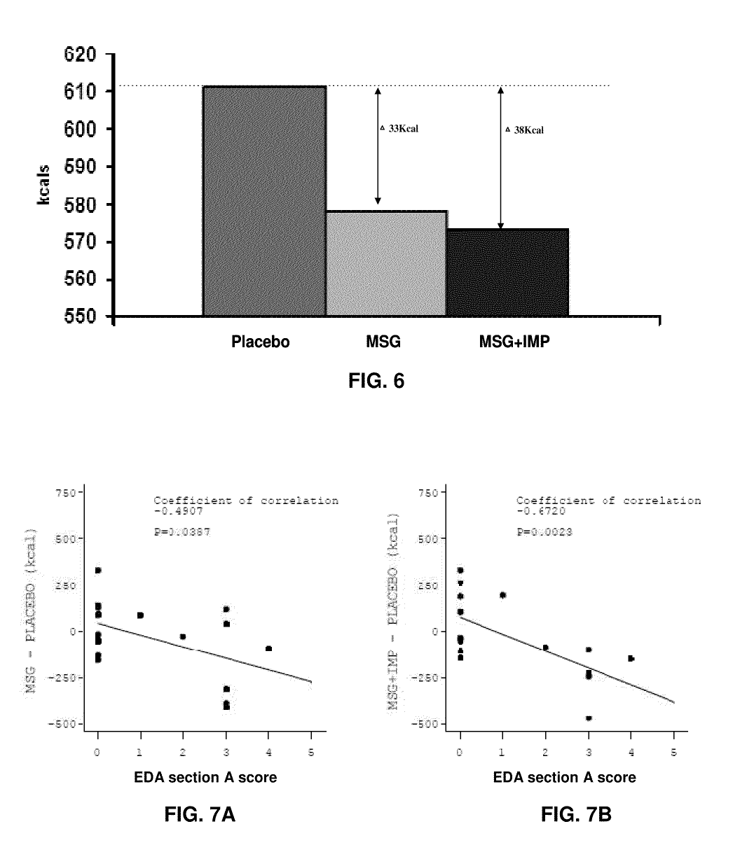

FIG. 6 demonstrates that the addition of umami substances to the soup decreased calorie intake. Kcals were calculated according to the amounts of each snack consumed. In comparison to the placebo soup, the soup that contains MSG or MSG+IMP showed an inhibition on calorie intake. The data indicates that MSG provides a superior effect of satiety.

FIGS. 7A and 7 B demonstrate that umami substances showed a larger effect in the suppression of calorie intake in subjects with a higher degree of concern for dieting and body weight. The “Eating Disorder Inventory” (EDI) consists of questionnaires of three sections, A (Drive for Thinness), B (Bulimia), and C (Body Dissatisfaction), and these are central to anorexia nervosa. EDI assesses attitudes and/or behaviors related to eating and body weight. ‘Drive for Thinness’ (Section A) indicates excessive concern with dieting, preoccupation with weight, and entrenchment in an extreme pursuit of thinness (Garner D M, Olmstead M P, Polivy J., Development and validation of a multidimensional eating disorder inventory for anorexia nervosa and bulimia. INT'L Journal of Eating Disorder. 2(2): 15-34, 1983). EDI score was obtained at the screening visit. FIGS. 7A and 7B show significant (p=0.0387 for FIG. 7A, p=0.0023 for FIG. 7B) association between EDI Section A scoring & the effect of umami substances addition on calorie intake—the higher the EDI score, the more the effect on the suppression of calorie intake. The data indicates that MSG provides a superior effect of satiety especially for a person who has a drive for thinness.

Thus, methods embodying principles of the present invention can include the ingestion of umami substances by a person who is first identified as one who has drive for thinness as indicated by their EDI Section A score, e.g., the person has a non-zero EDI Section A score. Other exemplary embodiments include that the person, who will ingest the umami substances described herein, is first identified as a person who has one or more of the following characteristics: (1) who eats sweets and carbohydrates without feeling nervous; (2) who thinks about dieting; (3) who feels extremely guilty after overeating; (4) who is terrified of gaining weight; (5) who exaggerates or magnify the importance of weight; (6) who is preoccupied with the desire to be thinner; and/or (7) who gets worried about keeping gaining if gaining a pound. Yet other embodiments include that the person, who will ingest the umami substances described herein, is first identified as a person who agrees with at least one statement having a meaning substantially the same as at least one of following statements: (1) I eat sweets and carbohydrates without feeling nervous; (2) I think about dieting; (3) I feel extremely guilty after overeating; (4) I am terrified of gaining weight; (5) I exaggerate or magnify the importance of weight; (6) I am preoccupied with the desire to be thinner; and/or (7) If I gain a pound I get worried that I will keep gaining.

In yet further methods embodying principles of the present invention, umami substances described herein can be added to a solid or liquid food or drink to a concentration of between 0.0001% to 10% by weight as a free glutamic acid in said food or drink.

For example, in the case that the umami substance is MSG, MSG is added to a liquid food or drink and a nucleic acid flavoring compound such as 5′-inosinic acid, 5′-guanylic acid, and 5′-adenylic acid and salts thereof is also added; a concentration of 0.0001% to 3% is preferable.

For example, in the case that the umami substance is MSG, MSG is added to a liquid food or drink and the nucleic acid flavoring compound is not added; a concentration of 0.01% to 3% is preferable.

For example, in the case that the umami substance is MSG, MSG is added to a solid food and a nucleic acid flavoring compound such as 5′-inosinic acid, 5′-guanylic acid, and 5′-adenylic acid and salts thereof is also added; a concentration of 0.0001% to 10% is preferable.

For example, in the case that the umami substance is MSG, MSG is added to a solid food and the nucleic acid flavoring compound is not added; a concentration of 0.01% to 10% is preferable.

A concentration of the nucleic acid flavoring compound can also be 0.001% to 1%.

As described above, through this invention, it is possible to provide agents for the inhibition, suppression, or prevention of overeating that have the effect of amplifying or sustaining a sense of fullness and will inhibit appetite stimulation that arises regardless of a sense of fullness. Agents for the inhibition, suppression, or prevention of overeating embodying principles of the present invention will be useful in the treatment of binge eating and in preventing and remedying obesity.

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

We claim:

1. A method for suppressing overeating, said method comprising ingesting at least one substance selected from the group consisting of glutamic acid and a salt thereof, in an amount sufficient to suppress overeating, by a person who needs suppression of overeating.

2. The method according to claim 1, wherein said sufficient amount for suppressing overeating is between 0.01 g to 100 g as a free glutamic acid, per ingestion for an adult human.

3. The method according to claim 1, further comprising:
adding between 0.01 g to 100 g of said substance per ingestion to a food or drink;
wherein said ingesting comprises ingesting said food or drink.

4. The method according to claim 3, wherein said adding comprises adding said substance to a concentration of between 0.0001% to 10% by weight as a free glutamic acid in said food or drink.

5. The method according to claim 1, wherein said person is a person who has drive for thinness as indicated by their EDI Section A score.

6. The method according to claim 1, wherein said person has a non-zero EDI Section A score.

7. The method according to claim 1, further comprising identifying said person prior to said ingesting as a person who agrees with at least one statement having a meaning substantially the same as at least one of following statements:
(1) I eat sweets and carbohydrates without feeling nervous;
(2) I think about dieting;
(3) I feel extremely guilty after overeating;
(4) I am terrified of gaining weight;
(5) I exaggerate or magnify the importance of weight;
(6) I am preoccupied with the desire to be thinner; and
(7) If I gain a pound I get worried that I will keep gaining.

8. The method for controlling or improving obesity, the method comprising suppressing overeating according to the method of claim 1.

9. The method for suppressing overeating by a patient who overeats, the method comprising prescribing to the patient medical food comprising:
(A) at least one substance selected from the group consisting of glutamic acid and a salt thereof; or
(B) a food or drink to which at least one substance selected from the group consisting of glutamic acid and a salt thereof has been added.

10. The method according to claim 9, wherein said medical food is:
(A) 0.01 g to 100 g of at least one substance selected from the group consisting of glutamic acid and a salt thereof, as a free glutamic acid, per ingestion by a adult human; or
(B) a food or drink to which 0.01 g to 100 g of at least one substance selected from the group consisting of glutamic acid and a salt thereof has been added, as a free glutamic acid, per ingestion by an adult human.

11. The method according to claim 9, wherein said medical food comprises said substance in a concentration of 0.0001% to 10% by weight as a free glutamic acid.

12. The method according to claim 9, wherein said prescribing comprises prescribing said medical food to a person who has drive for thinness as indicated by their EDI Section A score.

13. The method according to claim 9, wherein said person has a non-zero EDI Section A score.

14. The method according to claim 9, further comprising identifying said patient prior to said prescribing as a person who agrees with at least one statement having a meaning substantially the same as at least one of the following statements:
(1) I eat sweets and carbohydrates without feeling nervous,
(2) I think about dieting,
(3) I feel extremely guilty after overeating,
(4) I am terrified of gaining weight,
(5) I exaggerate or magnify the importance of weight,
(6) I am preoccupied with the desire to be thinner, and
(7) If I gain a pound I get worried that I will keep gaining.

15. A method for controlling or improving obesity, said method comprising suppressing overeating according to the method of claim 9.
